(19) United States
(12) Patent Application Publication
(10) Pub. No.: US 2018/0135187 A1
(43) Pub. Date: May 17, 2018

(54) Corrosion Inhibition

(71) Applicant: Schlumberger Technology Corporation, Sugar Land, TX (US)

(72) Inventors: Lynne Patricia Crawford, Cambridge (GB); Evgeny Borisovich Barmatov, Cambridge (GB); Trevor Lloyd Hughes, Cambridge (GB); Man Yi Ho, Cambridge (GB)

(21) Appl. No.: 15/566,529

(22) PCT Filed: Oct. 19, 2015
(86) PCT No.: PCT/US2015/056134

§ 371 Date: Oct. 13, 2017

(30) Foreign Application Priority Data
Apr. 13, 2015 (GB) 1506217.7

(51) Int. Cl. C23F 11/04; C23F 11/14; C07D 215/10; C07D 215/20; C07D 221/12; C09K 8/54; E21B 41/02

(52) U.S. Cl. CPC C23F 11/04; C23F 11/149; C07D 215/10; E21B 43/25; C07D 221/12; C09K 8/54; E21B 41/02; C07D 215/20

(57) ABSTRACT

Corrosion of steel in acidic solution is inhibited using a mixture of a compound in which a plurality of heterocyclic aromatic groups with a quaternary nitrogen atom are connected together, such as by a linking group which may be covalently attached to the quaternary nitrogen atoms of the aromatic heterocyclic rings, and a polarizable adsorption-intensifying anion.

### BACKGROUND

There are numerous circumstances where it is desired to protect metal, notably steel or an alloy steel, from corrosion. These include the protection of steel used in a subterranean borehole to access a hydrocarbon reservoir and the protection of steel exposed to a corrosive aqueous acidic liquid such as when steel tubing is used to convey a flow of acidic aqueous liquid.

One industry which has a need for protection of steel against corrosion is oil and gas exploration and production. Steel tubulars used in a borehole may be exposed to aqueous fluid for an extended period of time and fluid may contain or become mixed with corrosive solutes. More specifically, steel tubulars in a borehole and steel pipelines used to carry produced oil and gas may be exposed to acidic conditions. Exposure to acidic solution may be exposure to subterranean brine which happens to be acidic, or it may be exposure to acid deliberately used in an oilfield operation.

The technique of matrix acidizing in which the producing formation is treated with acid to stimulate production involves deliberate exposure of borehole steel to acid. This operation may be performed with coiled tubing which is run into a borehole and then used to convey acid down the borehole to the formation. When the operation comes to an end, the steel casing in the borehole and the exterior of the coiled tubing can be exposed to so-called unspent acid flowing back with formation fluids towards the surface.

Steel may be protected against corrosion by contacting the steel with an organic corrosion inhibitor. These organic inhibitors adsorb on the metal surface. Adsorbed inhibitor(s) may influence the rate of corrosion by one or more of several mechanisms: (i) by forming a physical barrier film which restricts the diffusion of species to/from the metal surface, (ii) by blocking anodic and/or cathodic reaction sites directly, (iii) by interacting with corrosion reaction intermediates adsorbed on the surface and (iv) by influencing the electrical double layer that forms at the metal/solution interface.

Adsorption may be physi-sorption which is the result of electrostatic attractive forces between inhibiting organic ions or dipoles and the electrically charged surface of the metal.

The surface charge of the metal is due to the electric field at the outer Helmholtz plane of the electrical double layer existing at the metal/solution interface.

Another possibility is that adsorption is by chemi-sorption, which takes place more slowly than electrostatic adsorption and with a higher activation energy. Chemi-sorption involves electron transfer from electron-rich sites within the structure of the inhibitor molecule(s) to vacant low energy orbitals in the metal. Typically, such electron-rich sites within an inhibitor molecule are heteroatoms with lone pair(s) of electrons or are multiple bonds and aromatic rings so that covalent bonds have electrons in n-orbitals. Because activation energy is required to bring about chemi-sorption, the extent of chemi-sorption and therefore the efficacy of corrosion inhibition may increase with temperature.

Aromatic nitrogen salts have been extensively used as corrosion inhibitors for mineral acids. Examples are n-alkyl pyridinium halides, n-alkylbenzylpyridinium halides, n-alkylisoquinolinium halides and n-alkylbenzylquinolinium halides. These compounds contain a cationic pyridinium or quinolinium group.

Corrosion inhibitors may be marketed as a mixture containing materials which inhibit corrosion together with so-called “intensifier” materials which enhance the inhibition of corrosion in various circumstances, even though these other materials do not function as corrosion inhibitors (or are less efficacious) if used alone. A number of materials have been found to act as intensifiers, including formic acid, methyl formate, potassium iodide and salts of copper, antimony and bismuth.

It is generally desirable to minimise corrosion and therefore desirable that a corrosion inhibitor should be effective. In some circumstances, it is also desirable to minimise the amount of corrosion inhibitor which is included in the corrosive solution, either on grounds of cost or because of apprehension that it will cause problems when the corrosive solution is used or further treated. Thus there is a desire for corrosion inhibitors which are effective at low concentration in the corrosive solution taking into account that all the exposed metal/alloy surfaces should be effectively protected

The corrosion inhibiting effect of an inhibitor or corrosion inhibiting mixture can be tested in various ways. One direct method of testing is to use a test piece which is a sample of the steel to be protected, customarily referred to as a “coupon”. This coupon is exposed for a measured length of time to an acidic solution containing a known concentration of corrosion inhibitor. The loss in weight of the coupon is measured and expressed as weight loss per unit surface area. The coupon may also be examined for localised pitting and the extent of pitting may be expressed as a numerical value: the so-called pitting index.

There are a number of other ways to measure corrosion by an acidic solution. These include linear polarization resistance measurement which was first proposed by M Stern and A L Geary in “Electrochemical Polarization: I. A Theoretical Analysis of the Shape of Polarization Curves” in J. Electrochem. Soc. Vol 104 pp 56-63 (1957) and followed by Stern: “A Method For Determining Corrosion Rates From Linear Polarization Data” in Corrosion, Vol. 14, No. 9, 1958, pp 440-444. In such tests a piece of the steel is used as an electrode and this electrode may be kept moving as a rotating disc, cylinder or cage to simulate flow of the corrosive solution over the steel.

Electrochemical Impedance Spectroscopy (EIS) can be used to monitor the corrosion rate of a system. The parameter of interest is the charge transfer resistance, Rct, which can be thought of the ‘resistance’ to corrosion. A metal test piece is used as the working electrode in a three-electrode electrochemical cell with the corrosive solution as electrolyte. Impedance data is obtained by applying a small alternating voltage to the working electrode and measuring the resulting current to obtain a value of impedance. This is done at a range of frequencies. Then, in order to extract the parameter of interest, the metal/inhibitor/electrolyte interfaces are modelled as an electrical equivalent circuit and the theoretical impedance is fitted to the data.

When steel is going to be exposed to a flow of a corrosive acidic composition, it is normal practice to test coupons of the steel with various concentrations of corrosion inhibitor in samples of the corrosive composition. A concentration of inhibitor which produces an acceptably low weight loss and pitting index is identified and this concentration of inhibitor is then maintained constantly in the flow of the corrosive composition to which the steel is exposed.

So-called stainless steels are alloy steels containing chromium and nickel as the main alloying metals. Alloy steels may have a single phase, either ferrite or austenite, or may have two phases mingled together.

The relative resistance of a stainless steel to chloride pitting and crevice corrosion can be related to alloy composition by the empirical formula known as the pitting resistance equivalent number (PREN). The most commonly used PREN expression is:

PREN=wt % Cr+3.3(wt % Mo+0.5(wt % W))+x·wt % N

where x is given as either 16 or 30.

Duplex stainless steels are composed of a mixture of austenite and ferrite phases, most typically with each phase in the range 25-75 vol %. They may have either a ferrite matrix or an austenitic matrix. As shown by the following table, the specified compositions for a range of duplex stainless steels include molybdenum and sometimes include other alloying metals such as tungsten, manganese and copper.

DUPLEX STAINLESS STEEL GRADES AND FEATURES

| | | Composition (wt %) | | | | | | | |
|---|---|---|---|---|---|---|---|---|---|
| Name | UNS no. | C | N | Cr | Ni | Mo | Cu, W, other | Ni/Cr ratio | PREN |
| “Lean” | S32101 | 0.03 | 0.22 | 21.5 | 1.5 | 0.3 | | 0.07 | 26 |
| 2304 | S32304 | 0.02 | 0.10 | 23.0 | 4.8 | 0.3 | | 0.21 | 26 |
| 2404 | S82441 | 0.02 | 0.27 | 24.0 | 3.6 | 1.6 | | 0.15 | 34 |
| 2205 | S32205 | ≤0.03 | 0.08-0.20 | 21.0-23.0 | 4.5-6.5 | 2.5-3.5 | Si ≥1.0 | 0.25 | 34 |
| | | | | | | | Mn ≥2.0 | | |
| 2507 | S32750 | 0.02 | 0.27 | 25.0 | 7.0 | 4.0 | | 0.28 | 43 |
| SM22Cr | | ≤0.03 | 0.08-0.20 | 21.0-23.0 | 4.5-6.5 | 2.5-3.5 | — | 0.25 | 34 |
| SM25Cr | | ≤0.03 | 0.10-0.30 | 24.0-26.0 | 5.5-7.5 | 2.5-3.5 | W 0.1-0.5 | 0.26 | 39 |
| SM25Cr W | | ≤0.03 | 0.24-0.32 | 24.0-26.0 | 6-8 | 2.5-3.5 | W 2.1-2.5 | 0.28 | 43 |
| DP3 | S31260 | 0.03 | 0.1-0.3 | 24.0-26.0 | 5.5-7.5 | 2.5-3.5 | Cu 0.2-0.8 | 0.26 | 39 |
| | | | | | | | W 0.1-0.5 | | |
| 255 | S32550 | 0.04 | 0.1-0.25 | 24.0-27.0 | 4.5-6.5 | 2.9-3.9 | Cu 1.5-2.5 | 0.22 | 40 |
| 100 | S32760 | 0.03 | 0.2-0.3 | 24.0-26.0 | 6.0-8.0 | 3.0-4.0 | Cu 0.5-1.0 | 0.28 | 41 |
| | | | | | | | W 0.5-1.0 | | |
| 52N+ | S32520 | 0.03 | 0.2-0.35 | 24.0-26.0 | 5.5-8.0 | 3.0-5.0 | Cu 0.5-3.0 | 0.27 | 43 |
| 2507 | S32507 | 0.03 | 0.24-0.32 | 24.0-26.0 | 6.0-8.0 | 3.0-5.0 | Cu 0.50 | 0.28 | 43 |

Typically, the duplex alloy 2205, contains 45-55 vol % austentite in a ferrite matrix, i.e. the ferrite is the continuous phase. The main alloying elements, chromium, molybdenum, nickel, manganese and nitrogen are not equally distributed in the two phases. Austentite is enriched in nickel, manganese and nitrogen whilst ferrite is enriched in chromium and molybdenum. The nickel-rich austenite phase is cathodic relative to the anodic ferrite matrix. Steel pipework and steel casing in a borehole are sometimes made of duplex stainless steel and are examples of duplex stainless steel which may be exposed to acidic solutions during well invention operations such as matrix acidizing treatments.

Other alloy steels have a single phase, which may be ferritic or autenitic. Examples are given in the following table:

SINGLE PHASE STAINLESS STEEL GRADES AND FEATURES

| | | Composition (wt %) | | | | | | | |
|---|---|---|---|---|---|---|---|---|---|
| | UNS | | | | | | Cu, W, | Ni/Cr | |
| Name | no. | C | N | Cr | Ni | Mo | other | ratio | PREN |

AUSTENITIC

| | | | | | | | | | |
|---|---|---|---|---|---|---|---|---|---|
| 254 | S31254 | 0.02 | 0.18-0.22 | 19.5-20.5 | 17.5-18.5 | 6.0-6.5 | Cu 0.50-1.0 | 0.9 | 44 |
| SMO | | | | | | | | | |
| Nirosta | S24565 | 0.03 | 0.4-0.6 | 23.0-25.0 | 16.0-18.0 | 3.5-5.0 | Mn 3.5-6.5 | 0.708 | 46 |
| 456SS | | | | | | | | | |
| 654- | S32654 | 0.02 | 0.45-0.55 | 24.0-26.0 | 21.0-23.0 | 7.0-8.0 | Cu 0.3-0.6 | 0.88 | 58 |
| SMO | | | | | | | Mn 2.0-4.0 | | |

FERRITIC

| | | | | | | | | | |
|---|---|---|---|---|---|---|---|---|---|
| E-brite | S44627 | 0.01 | 0.015 | 25.0-27.0 | 0.50 | 0.75-1.50 | Cu 0.20 | 0.019 | 30 |
| 26-1 | | | | | | | Nb 0.05-0.2 | | |
| Monit | S44635 | 0.025 | 0.035 | 24.5-26.0 | 3.5-4.5 | 3.5-4.5 | — | 0.158 | 39 |
| Seacure | S44660 | 0.03 | 0.040 | 25.0-28.0 | 1.0-3.5 | 3.0-4.0 | — | 0.085 | 39 |
| AL | S44735 | 0.03 | 0.045 | 28.0-30.0 | 1.00 | 3.6-4.2 | — | 0.034 | 43 |
| 29-4C | | | | | | | | | |

### SUMMARY

This summary is provided to introduce a selection of concepts that are further described below. This summary is not intended to be used as an aid in limiting the scope of the subject matter claimed.

A first aspect of the present disclosure is a method of inhibiting corrosion of metal exposed to aqueous acidic solution where the method comprises providing in the solution: at least one inhibitor compound comprising two to five groups which are connected together, each of the groups containing a heterocyclic aromatic ring or ring system comprising at least one ring nitrogen atom which is quaternary in the aqueous acidic solution, and at least one polarizable adsorption-intensifying anion which comprises at least one atom of atomic number 52 or above and/or an anionic group conjugated with an aromatic ring or ring system, the concentration, in equivalents, of said anions being at least equal to the concentration, in equivalents, of the quaternary nitrogen atoms.

Optionally, the at least one ring nitrogen atom may be tertiary in aqueous neutral or alkaline solutions.

We have found that compounds comprising such a plurality of groups which contain quaternary nitrogen and are connected together are effective as corrosion inhibitors, but are much more effective when a polarizable adsorption intensifier is present. The adsorption intensifier is a polarizable anion. It may be polarizable because the anion comprises an atom of sufficiently high atomic number or because an anionic group is conjugated with an aromatic ring or ring system so that there is a large molecular orbital.

A polarizable anion containing an atom of atomic number 52 or above may be iodide ion. Iodine has atomic number 53. Another possible polarizable anion is the triiodide ion containing three iodine atoms with a single negative charge, which can be formed by reaction of iodine and iodide ions and is more easily polarizable than iodide ion itself. Examples of polarizable anion in which an anionic group is conjugated to an aromatic ring system include para-toluene sulphonate (tosylate) and naphthalene sulphonate.

The concentrations of the compounds should be such that the polarizable anions are at least equivalent to the quaternary nitrogen atoms of the inhibitor compounds. This is stated by requiring the concentration of polarizable anions, in equivalents per unit volume, to be equal to the concentration of quaternary nitrogen atoms, in equivalents in the same unit volume.

To illustrate, assume there is one inhibitor compound containing two heterocyclic ring systems, each with one nitrogen atom. The concentration of the inhibitor compound is 1 millimole per litre. The equivalent concentration of polarizable anions with a single negative charge such as iodide or tosylate would be 2 millimole per litre. If a polarizable anion with a double negative charge were to be used, the equivalent concentration would be 1 millimole per litre.

The concentration of the polarizable anion or anions may be more than the amount required above. The concentration, in equivalents, of polarizable anions may at least twice or at least 3 times the concentration, in equivalents, of the quaternary nitrogen atoms. It is possible that the molar concentration of polarizable anion is at least equal to the concentration, in equivalents, of the quaternary nitrogen atoms: this would make a difference if the polarizable anion carried more than a single negative charge. Of course, iodide, triodide and suphonates as mentioned above all carry a single negative charge.

The number of quaternary nitrogen-containing groups in an inhibitor compound may be from two to four and more specifically it may be two or three. One, some or all of the quaternary nitrogen-containing groups in an inhibitor compound may be connected to the others through the group's quaternary nitrogen atom. Connection of one group to the others through its quaternary nitrogen atom offers a synthetic route in which a reaction that connects the group can also quaternise the nitrogen atom. The plurality of nitrogen-containing groups may be connected together by attachment to a linking moiety which may be an atom or may be a linking group containing a plurality of atoms, such as a chain of at least three carbon atoms. In some embodiments all of the nitrogen-containing groups are connected to each other through their quaternary nitrogen atoms.

The heterocyclic aromatic ring or ring system of a nitrogen-containing group may be such that it contains only a single nitrogen atom and this single nitrogen atom is quaternary. Such a ring system may be convenient as a structure for use in making a compound as specified above.

The term “quaternary” denotes a nitrogen atom bearing positive charge and which can be depicted as having four covalent bonds to atoms other than hydrogen. For example, tetramethyl ammonium bromide having four carbon to nitrogen bonds in a structure

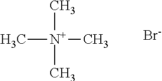

Another example is N-methyl pyridinium bromide which can be drawn as having a single bond and a double bond to ring carbon atoms and a single bond to the methyl group, in the structure

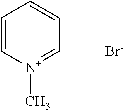

These are both compounds containing quaternary nitrogen. The quaternary nitrogen atom does not have a covalent bond to hydrogen. Of course, it is appreciated that drawing an aromatic ring with single and double bonds is a convenient device which reflects the number of bonds to a nitrogen atom without showing the delocalisation of electrons in aromatic rings.

The at least one ring nitrogen atom is quaternary in the aqueous acidic solution. The at least one ring nitrogen atom may be permanently positively charged, as shown in the above examples.

Alternatively the at least one ring nitrogen atom may be positively charged only in aqueous acidic solutions and deprotonate to become a neutral nitrogen atom in neutral and alkaline solutions. This has the advantage that, because the inhibitor is neutral and thus insoluble in neutral and alkaline solutions, toxicity of the inhibitor is reduced.

The term “tertiary” denotes a nitrogen atom bearing no charge and which can be depicted as having three covalent bonds to atoms other than hydrogen. Some examples are shown below:

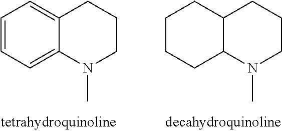

The neutral tertiary nitrogen in the present disclosure protonates in acidic solutions to become a positively charged quaternary nitrogen:

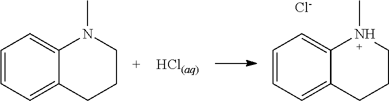

We have found that a structure in which a plurality of quaternary nitrogen-containing groups are linked can be more effective as a corrosion inhibitor than a molecule containing a single such group. A linking group containing carbon and hydrogen atoms can serve to make the molecule more hydrophobic, which can also enhance effectiveness as a corrosion inhibitor. This effectiveness can then be enhanced further by the presence of polarizable anions.

Some structures of inhibitor compound as above have all the nitrogen-containing groups covalently bonded to a single linking moiety, but other structural configurations such as a chain of the nitrogen-containing groups connected one to the next are also possible. Compounds with a single linking moiety may have a structure which can be represented by a general formula

YB)ₘ

where B denotes a heterocyclic aromatic ring or ring system which contains a plurality of carbon atoms and at least one nitrogen atom which is quaternary, Y is the linking moiety, i.e. an atom or group to which the groups B are attached, and m is two to five. Bonds within a group B and bonds between a group B and linking moiety Y may all be covalent bonds.

The plurality of nitrogen-containing heterocyclic groups (groups B in the formula above) may all be the same or may be a mixture of different heterocyclic groups.

It is possible that Y could be a single atom to which a plurality of nitrogen-containing groups B are attached. Where Y is a linking group containing more than one atom, it may contain a saturated carbon chain of at least three carbon atoms, providing some flexibility in the connection between groups B. A linking group Y may be a linear or branched hydrocarbon chain of at least three carbon atoms and may incorporate an olefinic unsaturation or an aromatic ring or rings.

In further aspects, the present disclosure provides each of:
An aqueous solution, which may be an aqueous acidic solution, having dissolved or suspended therein at least one inhibitor compound and at least one polarizable anion as defined according to the first aspect of this disclosure;
A corrosion inhibiting composition, which may be a liquid concentrate intended to be used as a corrosion inhibiting additive in an aqueous solution, containing at least one inhibitor compound and at least one polarizable anion in accordance with the first aspect of this disclosure and a carrier material which may be a carrier fluid;
Use of at least one inhibitor compound and at least one polarizable anion in accordance with the first aspect of this disclosure to inhibit corrosion during exposure to aqueous solution.

Some embodiments of the method disclosed here are a method of inhibiting corrosion of steel surfaces in a system exposed to an aqueous liquid, wherein the surfaces comprise at least two steels which differ in composition, i.e. differ qualitatively and/or quantitatively in the elements additional to iron which are present in the steel.

In some embodiments the method is a method of inhibiting corrosion of duplex steel surfaces in a system exposed to an aqueous liquid. The steel surfaces may then comprise at least two steels which differ in composition where at least one steel surface is a duplex steel.

The metal(s) which are or will be exposed to aqueous solution may located in a subterranean borehole. The metal to be protected from corrosion may be tubing along which the solution is pumped and which may be located in a subterranean borehole, connected to such a borehole, or positioned to deliver into such a borehole (as is the case when coiled tubing is in use). A wide range of different compositions of metal surfaces may be exposed to the aqueous liquid; such metals include carbon steels and low and high alloy steels.

Aqueous solution containing at least one inhibitor compound and at least one polarizable anion defined according to the first aspect of this invention may be acidic but it may possibly be a solution which will subsequently become acidic by mixing with acidic material. An acidic solution may possibly be a solution with pH below zero, as is the case with a solution used for acid stimulation of a well. Acid stimulation techniques include acid fracturing and matrix acidizing. Thus the corrosion inhibiting compound may be utilized in an acid stimulation composition and procedure, which may be a matrix acidizing composition and procedure.

### BRIEF DESCRIPTION OF DRAWINGS

FIG. 1 is a graph showing weight loss results obtained in Examples 1 and 2 below, with lb/ft² on the left hand axis and corresponding gm/m² at the right;

FIG. 2 is the equivalent circuit referred to in Example 6 below;

FIG. 3 is a graph showing results obtained in Example 6 below; and

FIG. 4 is a graph showing results obtained in Example 7 below.

### DETAILED DESCRIPTION

As indicated in the summary above, the present disclosure is concerned with mixtures in which there are one or more compounds which contain a plurality of groups each of which contains charged quaternary nitrogen. This disclosure is also concerned with the use of such mixtures as corrosion inhibitors in a corrosive solution, in particular in a corrosive acidic aqueous solution. Surfaces to be protected from corrosion will ordinarily be metallic and the metal may be an alloy. For instance surfaces to be protected may be steel and the steel may be a single phase steel or a duplex alloy steel. An alloy steel may contain nickel, chromium, molybdenum and possibly other alloying metals.

In some embodiments, such a quaternary nitrogen-containing compound is included in an inhibitor composition which is used to protect a system in which there are a plurality of metals which come into contact with corrosive aqueous acidic liquid. The metals may be steel or steel alloys and they may be located within a subterranean borehole.

Quaternary nitrogen in heterocyclic aromatic rings or fused ring systems is often written as having three bonds to other atoms of a ring. A pyridine ring is a single six membered ring containing one such nitrogen. It may be incorporated into a larger molecule through a covalent bond to its nitrogen atom, thereby quaternizing the nitrogen atom.

Thus the following reactions lead to instances of inhibitor compounds with a plurality of connected groups containing quaternary nitrogen in an aromatic ring:

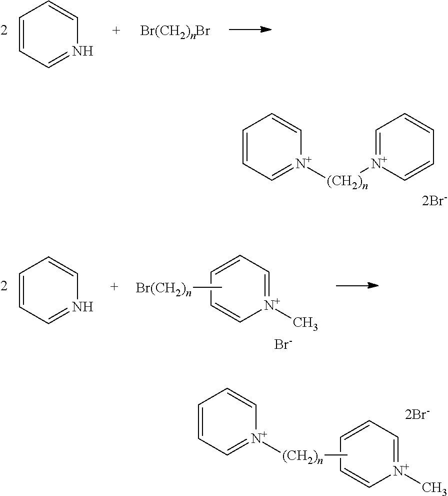

The products of both these reactions are possibilities within the abovementioned general formula

YB)ₘ

where in this case Y is the polymethylene group shown as (CH₂)ₙ, B is a pyridine ring and m is 2.

The above compound of formula

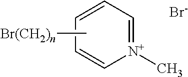

may be obtained by a synthetic route including synthesis of the pyridine ring with the polymethylene group attached.

Instances of fused aromatic ring systems which contain nitrogen atoms which can become quaternary when joined to a linking group are

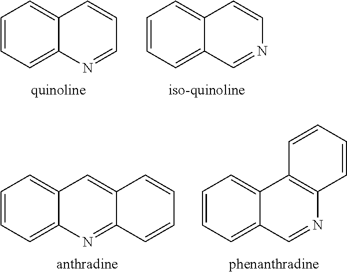

These fused ring systems can be connected by linking groups covalently attached to nitrogen atoms which then become quaternary. The reaction may be directly analogous to the reaction of two molecules of pyridine with a polymethylene dibromide as shown above.

Heterocyclic rings and rings systems may contain more than one nitrogen atom, as is the case with pyrimidine which has two ternary nitrogen atoms:

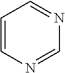

Purine consists of a pyrimidine ring fused to an imidazole ring and has three ternary nitrogen atoms and also a secondary nitrogen atom. Purine itself may be used as a nitrogen-containing group and so can other compounds with a purine structure, shown below:

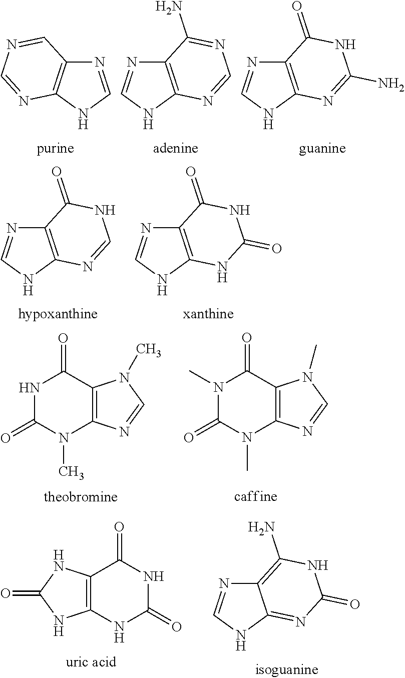

A heterocyclic aromatic ring may contain another hetero atom as well as nitrogen. An example is oxazole which contains one nitrogen atom and one oxygen atom in a five membered ring.

It is also possible that quaternary nitrogen-containing heterocyclic groups are attached by an unsaturated linking group attached to carbon atoms of heterocyclic rings. The following structure is an example of this in which the two nitrogen containing groups are quinolinium groups quaternized with naphthyl groups

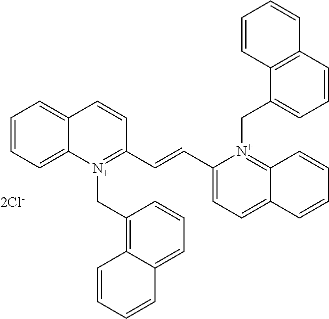 [SMILES: C(=C\c1ccc2ccccc2[n+]1Cc1cccc2ccccc12)/c1ccc2ccccc2[n+]1Cc1cccc2ccccc12]

A further possibility for the inhibitor compounds is that some or all of the nitrogen-containing groups have additional groups attached which are intended to enhance adsorption onto a metal surface or add further functionality. For instance a quinolinium group could have an electron-rich naphthylmethoxy group attached, as illustrated by the partial structure

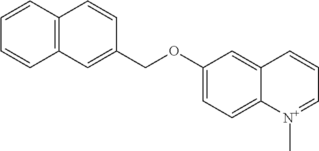 [SMILES: C[n+]1cccc2cc(OCc3ccc4ccccc4c3)ccc21]

A naphthyl methoxy group may be introduced by reaction of 1-(chloromethyl) naphthalene with 6-hydroxyquinoline as will be exemplified below.

A benzyloxy group could be attached in the same way as the naphthylmethoxy group above. These electron rich groups will be able to chemisorb to the metal surface, enhancing the protection of the surface.

Another possibility for an additional group attached to a nitrogen-containing heterocyclic ring is a styryl group. The styryl group may be introduced by reaction of benzaldehyde with a methyl substituent on a heterocyclic ring, as illustrated by the following reaction scheme leading to a structure in which styryl groups are attached to two quinolinium groups

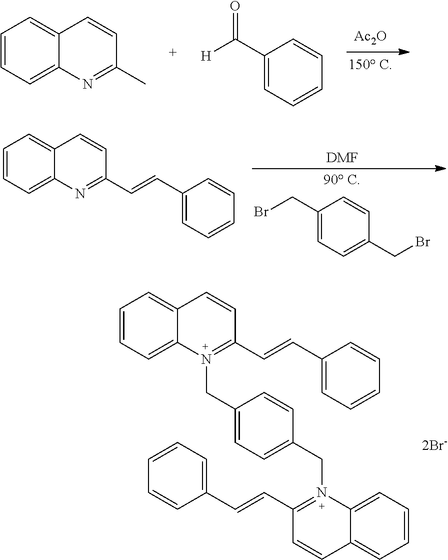

Optionally, the at least one ring nitrogen atom may be tertiary in aqueous neutral or alkaline solutions.

One dimer example is shown below:

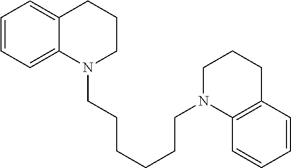 [SMILES: c1ccc2c(c1)CCCN2CCCCCCN1CCCc2ccccc21]

The ring nitrogen atoms in the above molecule protonate in aqueous acidic solutions and become quaternary nitrogen atoms. This provides solubility and thus allows the molecule to act as an acid corrosion inhibitor.

Optionally, further groups can be attached to the structure. A trimer example is shown below:

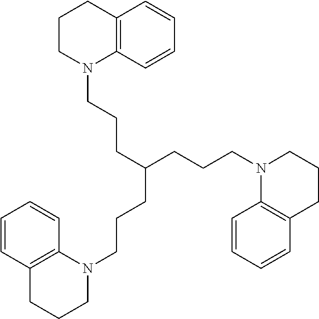 [SMILES: c1ccc2c(c1)CCCN2CCCC(CCCN1CCCc2ccccc21)CCCN1CCCc2ccccc21]

As already noted, a linking group which connects nitrogen-containing groups in an inhibitor compound as disclosed herein may contain a chain of carbon atoms. A linking group may be aliphatic or aromatic. Specific possibilities for a linking group joining two nitrogen-containing groups are a saturated polymethylene chain

—(CH₂)ₙ—

an unsaturated alkylene chain, i.e. a chain of aliphatic carbon atoms with at least one olefinic double bond in the chain such as

—CH₂—CH═CH—CH₂—

and a mixed aliphatic aromatic chain in which two aliphatic groups are connected by an aromatic ring, such as

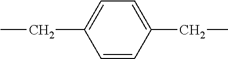

A linking group may join three or more nitrogen-containing groups. For joining three or more such groups a linking group could be a branched aliphatic structure such as

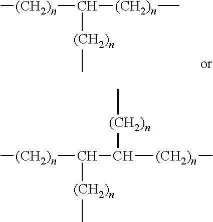

or could be a mixed aliphatic aromatic structure with methylene groups joined to an aromatic ring, for example

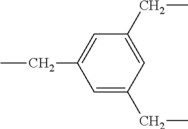

The examples of synthesis below mention two compounds in which three heterocyclic groups are attached to a linking group.

A further instance of such a compound is the structure:

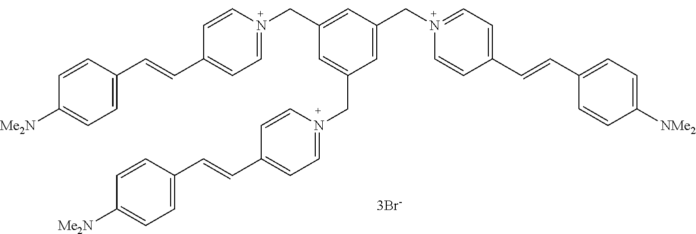

This has been disclosed as a dye, by Mishra and Haram in Dyes and Pigments (2004), 63(2), pp 191-202.

The linking groups referred to above would allow three or four nitrogen-containing groups to be connected by covalent bonding to a single linking group. In an alternative possibility, a plurality of groups are connected together as an oligomeric chain, for example as illustrated by the following structure:

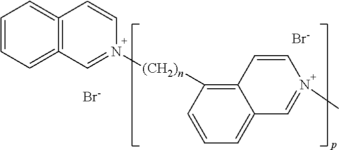

in which p is 1 to 4. This compound is an oligomer derivable from a bromoalkylisoquinoline

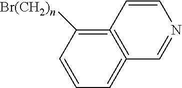

The inhibitor compounds described above are used in conjunction with a polarisable anion. The polarisable character may come about because the anion comprises an atom of atomic number 52 or more. Such an atom is large enough that some of its electrons occupy orbitals at distance from the nucleus. Iodine has atomic number 53 and iodide ion may be the most readily accessible ion of this character. The inhibitor compound may be provided as an iodide salt or it may be mixed with another iodide such as potassium iodide. If the inhibitor compound is provided as an iodide salt, additional iodide may be added to the mixture. Another possible anion is tri-iodide, which may be made by reacting iodine with potassium iodide in aqueous solution.

Polarisable character of an anion can also come about when an ion contains an anionic group such as sulfonate, attached to an aromatic ring or ring system. One anion which may be used is para-toluene methyl sulphonate, often referred to as tosylate, having the formula

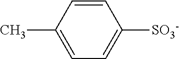

Another possibility is naphthalene sulphonate, having formula:

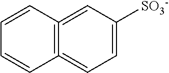

In addition, one or more of the two to five nitrogen-containing groups may have one or more additional groups attached which are intended to enhance adsorption onto a metal surface or provide an additional function:

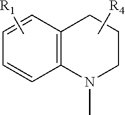

For instance, the group below could have an additional polymerisable group attached to one of its rings, as illustrated below:

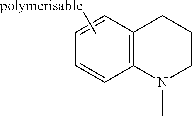

Possible polymerisable groups for example include —C(OH)—C≡CH, C≡C—, and/or —C≡N. Such a polymerisable group is intended to polymerise with other polymerisable groups after adsorption onto a metal surface, and this enables the molecules to combine together as a protective film which enhances acid corrosion inhibition.

### Examples of Synthesis of Inhibitor Compounds

A general method of synthesis of compounds in which a linking group connects to nitrogen atoms of the nitrogen containing groups is based on a paper by Hartwell and Pogorelskin, J.A.C.S. vol 72 pp 2040-2044 [1950].

A nitrogen containing compound (used with 20% excess) and a precursor of the linking group terminating in halogen atoms are reacted as a suspension in dimethylformamide (DMF) which is an aprotic solvent. The product is collected by addition of acetone and filtration.

A number of compounds were prepared by this general method and details of some preparations are given by way of example below.

### 1,1′-(Propane-1,3-diyl) di-quinolinium bromide

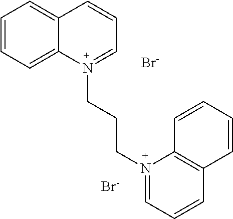

Quinoline (5 g, 38.7 mmol) and 1,3-dibromopropane (3.26 g, 16.1 mmol) were mixed together in N,N′-dimethylformamide (8 ml) as solvent and heated at 90° C. for 18 hr. The suspension was cooled, acetone was added, and the solid filtered off then washed with acetone and diethyl ether to give a solid product (6.63 g). The solid was crystallised from ethanol (32 ml), filtered, washed with ethanol and diethyl ether and dried to give 1,1′-(propane-1,3-diyl)diquinolinium bromide, 4.95 g (66%).

¹H NMR 400 MHz (DMSO_{d6}) δ=9.72 (d, 2H J=8 Hz), 9.34 (d, 2H, J=8 Hz), 8.90 (d, 2H, J=8 Hz), 8.53 (d, 2H J=8 Hz), 8.31 (bt, 2H J=8 Hz), 8.24 (dd, 4H J=8 Hz), 8.08 (t, 2H J=8 Hz), 5.41 (t, 4H J=8 Hz)

### 1,1′-(Propane-1,3-diyl) di-quinolinium iodide

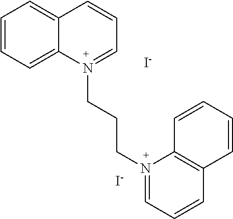

Quinoline (5 g, 38.7 mmol) and 1,3-diiodopropane (4.77 g, 16.1 mmol) were mixed together in N,N′-dimethylformamide (8 ml) and heated at 90° C. for 18 hr. The suspension was cooled and the solid filtered, washed with acetone and dried to give the product 1,1′-(propane-1,3-diyl)diquinolinium iodide, 7.3 g (81%) which was used without further purification.

¹H NMR 400 MHz (D₂O) δ=9.36 (d, 2H J=8 Hz), 9.17 (d, 2H J=8 Hz), 8.44 (d, 2H J=8 Hz), 8.41 (d, 2H J=8 Hz), 8.27 (t, 2H J=8 Hz), 8.07 (t, 2H J=8 Hz), 8.05 (t, 2H J=8 Hz), 5.39 (t, 4H J=8 Hz), 3.04 (bm, 2H)

### 1,1′-(Butane-1,4-diyl)diquinolinium bromide

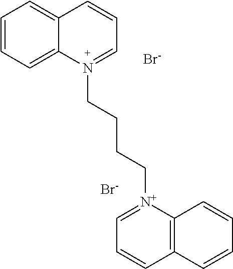

Quinoline (5 g, 38.7 mmol) and 1,4-dibromobutane (3.48 g, 16.1 mmol) were mixed together in N,N′-dimethylformamide (8 ml) and heated at 90° C. for 18 hr. The suspension was cooled, the solid filtered, washed with acetone and diethyl ether to give a solid. The solid was triturated with hot ethanol (80 ml), cooled, filtered washed with ethanol and diethyl ether and dried to give the product 1,1′-(butane-1,4-diyl)diquinolinium bromide 6.2 g (81% yield) which was used without further purification.

¹H NMR 400 MHz (D₂O) δ=9.55 (d, 2H J=8 Hz), 9.31 (d, 2H J=8 Hz), 8.70 (d, 2H J=8 Hz), 8.52 (d, 2H J=8 Hz), 8.31 (t, 2H J=8 Hz), 8.18 (dd, 2H J=8 Hz), 8.08 (t, 2H J=8 Hz), 5.12 (bs, 4H), 2.28 (bs, 4H)

### (E)-1,1′-(But-2-ene 1,4-diyl)diquinolinium bromide

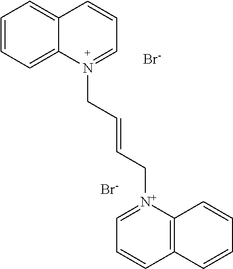

Quinoline (3.1 g, 24 mmol) and 1,4-dibromobut-2-ene (2.14 g, 10 mmol) were mixed together in N,N′-dimethylformamide (8 ml) and heated at 90° C. for 18 hr. The suspension was cooled, the solid filtered, washed with acetone and diethyl ether to give the product (E)-1,1′-(but-2-ene 1,4-diyl)diquinolinium bromide, 4.3 g (92% yield) which was used without further purification.

### 1,1′-(1-4-phenylenebis(methylene)diquinolinium bromide

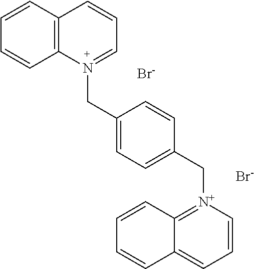

Quinoline (1.52 g, 11.6 mmol) and 1,4-bis(bromomethyl)benzene (1.3 g, 4.9 mmol) were combined in N,N′-dimethylformamide (4 ml) and heated at 90° C. for 18 hr. The suspension was cooled, the solid filtered, washed with acetone, diethyl ether and dried to 1,1′-(1-4-phenylenebis(methylene) diquinolinium bromide, 2.4 g (93% yield) which was used without further purification.

### 6-(Naphthalen-1-ylmethoxy)-1-(3-(6-naphthalen-2-ylmethoxy) quinolinium-1-yl) propyl) quinolinium bromide (14)

This compound was prepared in two stages, by the following reaction scheme.

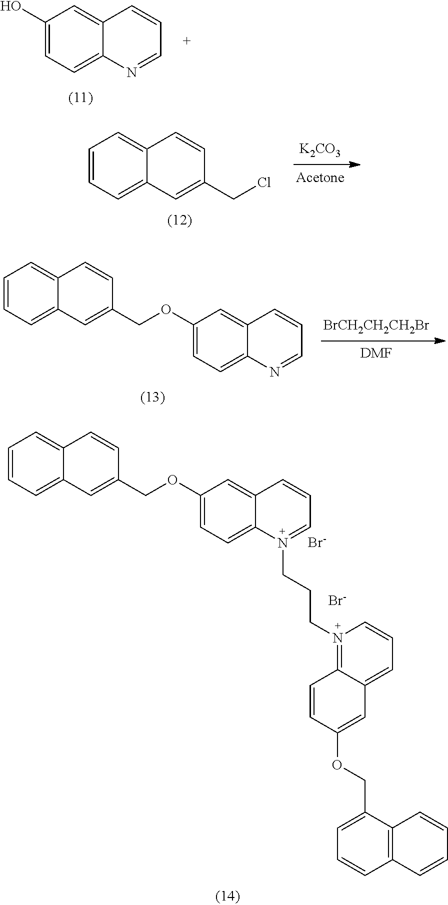

In the first stage, 6-Hydroxyquinoline (11) (5 g, 34.4 mmol) was added to acetone (200 ml) and heated to just below reflux. Potassium carbonate (24 g, 0.17 mol) was added portion wise and heated at reflux for 5 min. 1-(Chloromethyl) naphthalene (12) (6.07 g, 34 mmol) was added portion wise and the suspension heated at reflux for 23 hr. The suspension was cooled, the solid filtered and washed with acetone and the filtrated evaporated. The crude product was purified by column chromatography eluting with 0-10% methanol in dichloromethane. The combined column fractions were evaporated and the residue triturated with hexane to give a solid which was filtered and washed with hexane to give 6-(naphthalen-2-ylmethoxy) quinoline (13), 6.7 g (68% yield).

In the second stage, 6-(Naphthalen-2-ylmethoxy) quinoline (13) (1.3 g, 4.55 mmol) and 1,3-dibromopropane (402 mg, 1.99 mmol) were added to in N,N′-dimethylformamide (2 ml) and heated at 90° C. for 22 hr. The suspension was cooled and the solid filtered, washed with acetone, diethyl ether and dried to give 6-(naphthalen-1-ylmethoxy)-1-(3-(6-naphthalen-2-ylmethoxy) quinolinium-1-yl) propyl) quinolinium bromide (14), 1.35 g (87% yield) which was used without further purification.

### Tri-Quinolinium Inhibitors

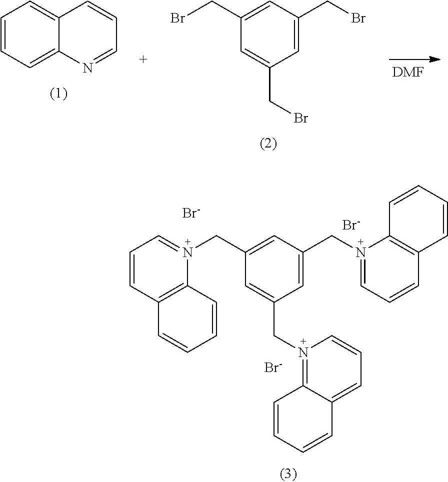

Quinoline (1) in small excess was reacted with 1,3,5-tris(bromomethyl)benzene (2) in dimethylformamide with procedure as before to form the trisquinolinium species (3), 8.9 g (100% yield). An aliquot of the trisquinolinium bromide species (3) was dissolved in methanol and KI added. The resultant solid (iodide salt) was filtered off and dried

In the same way quinoline (1) was reacted with 2,4,6-tris(bromomethyl)mesitylene (4) to obtain the trisquinolinium species (5), 8.3 g (84% yield).

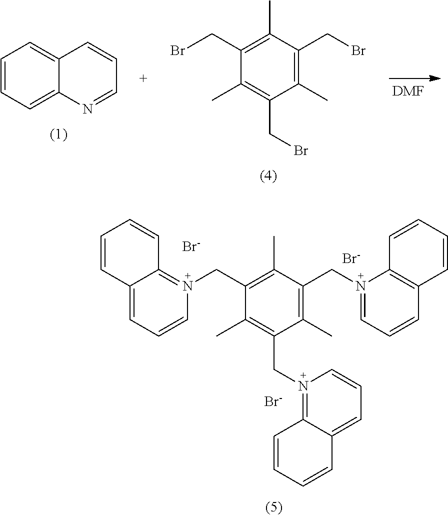

### Tri-Phenanthridinium Inhibitor

The same procedure was used to react phenanthridine (6) with 1,3,5-tribromomethylbenzene (2) to obtain the trisphenanthridium species (7), 5.0 g (94% yield).

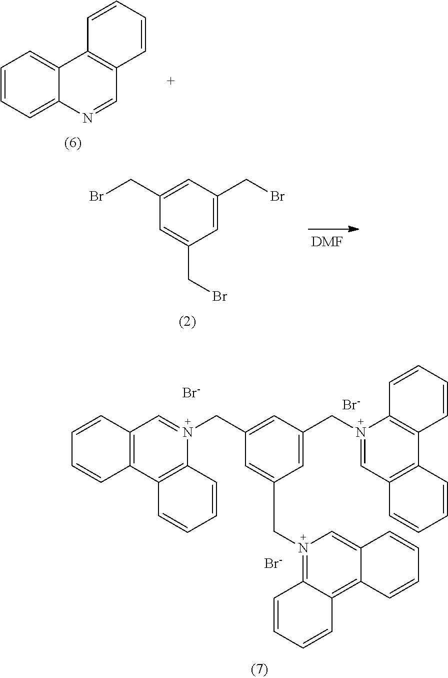

### EXPERIMENTAL EXAMPLES

Experiments were carried out with coupons of the following steels:
HS80, a low carbon steel used to fabricate coiled tubing.
N80, a medium carbon steel used to fabricate borehole casing.
13Cr80, an alloy steel containing chromium without nickel, also used to fabricate borehole casing.
22Cr125 also designated 2205, a duplex alloy steel which is an iron-chromium-nickel-molybdenum alloy, also used (among other things) to fabricate casing.

### Example 1

Experiments were carried out to observe corrosion rates on coupons of the steels above. Corrosion test coupons with surface area of 25-30 cm² were glass bead blasted to ensure a clean and homogeneous surface, measured to determine their exact surface area, weighed and then exposed to 4 molar hydrochloric acid solution in a continuously stirred corrosion cell containing 200 mL acid solution per test coupon. The temperature was held at 78° C. with the acid solution pre-heated to this temperature before immersing the test coupon. The acid solution contained a corrosion inhibiting material or mixture of materials. After a test period of three hours, the coupons were removed from the solution, washed and dried and re-weighed so as to determine the weight loss. Results were expressed as weight loss per unit area.

This test procedure was used to compare corrosion inhibition in an acid solution which contained 5 mM hexanediyl di-quinolinium bromide (a quinolinium dimer embodying the concepts disclosed herein) in the presence of 20 mM potassium iodide. The tests on 22Cr 125 steel also included a comparative test of corrosion inhibition in a solution which contained 10 mM propyl-quinolinium bromide (twice the concentration because it is a mono-quinolinium compound) again in the presence of 20 mM potassium iodide. The measurements of weight loss per unit area, over the three hour test period, are given in the following table.

| | | Total | Weight | Weight |
|---|---|---|---|---|
| | | iodide | loss | loss |
| Inhibitor in acid solution | Steel | conc. | (lbs/ft²) | (gm/m²) |
| 5 mM hexanediyl di- | HS80 | 20 mM | 0.013 | 63.4 |
| quinolinium bromide | | | | |
| 5 mM hexanediyl di- | N80 | 20 mM | 0.008 | 39.0 |
| quinolinium bromide | | | | |
| 5 mM hexanediyl di- | 13Cr | 20 mM | 0.032 | 156 |
| quinolinium bromide | | | | |
| 5 mM hexanediyl di- | 22Cr125 | 20 mM | 0.005 | 24.4 |
| quinolinium bromide | | | | |
| 10 mM propyl mono- | 22Cr125 | 20 mM | 0.019 | 92.7 |
| quinolinium bromide | | | | |

It is apparent that weight loss was lowest on 22Cr125 steel and that on this steel the di-quinolinium compound gives much improved corrosion inhibition as compared to the comparative mono-quinolinium compound.

A similar comparison was made using decanediyl di-quinolinium bromide (another quinolinium dimer embodying the concepts disclosed herein) in the presence of added potassium iodide and isopentyl mono-quinolinium iodide in the presence of some additional iodide provided as potassium iodide.

The following table gives the concentrations of the inhibitor molecules, the total concentration of iodide in solution (introduced both as counterion of the inhibitor and as potassium iodide) and the measurements of weight loss per unit area over the three hour test period.

| | | Total | Weight | Weight |
|---|---|---|---|---|
| | | iodide | loss | loss |
| Inhibitor in acid solution | Steel | conc. | (lbs/ft²) | (gm/m²) |
| 2 mM decanediyl di- | 22Cr125 | 16 mM | 0.0062 | 30.2 |
| quinolinium bromide | | | | |
| 5 mM isopentyl mono- | 22Cr125 | 15 mM | 0.0098 | 47.8 |
| quinolinium iodide | | | | |
| 5 mM isopentyl mono- | 22Cr125 | 25 mM | 0.0064 | 31.2 |
| quinolinium iodide | | | | |
| 5 mM decanediyl di- | HS80 | 20 mM | 0.0059 | 28.8 |
| quinolinium bromide | | | | |
| 5 mM decanediyl di- | N80 | 20 mM | 0.0045 | 22.0 |
| quinolinium bromide | | | | |
| 5 mM decanediyl di- | 13Cr | 20 mM | 0.0090 | 43.9 |
| quinolinium bromide | | | | |
| 5 mM decanediyl di- | 22Cr125 | 20 mM | 0.0044 | 21.5 |
| quinolinium bromide | | | | |

The first three rows in the table above show that 2 mM decanediyl di-quinolinium bromide with 16 mM iodide matched the efficacy of 5 mM isopentyl mono-quinolinium iodide with 25 mM iodide, even though the diquinolinium compound was used at a concentration such that the concentration of quaternary nitrogen atoms was slightly less than with the monoquinolinium compound, and the concentration of iodide was also lower.

### Example 2

Weight loss testing as in the previous example was carried out on 22Cr125 steel with 5 mM concentrations of diquinolinium compounds listed below and with varying concentrations of potassium iodide in solution. The diquinolinium compounds were
propanediyl diquinolinium bromide
butanediyl diquinolinium bromide
hexanediyl diquinolinium bromide
decanediyl diquinolinium bromide

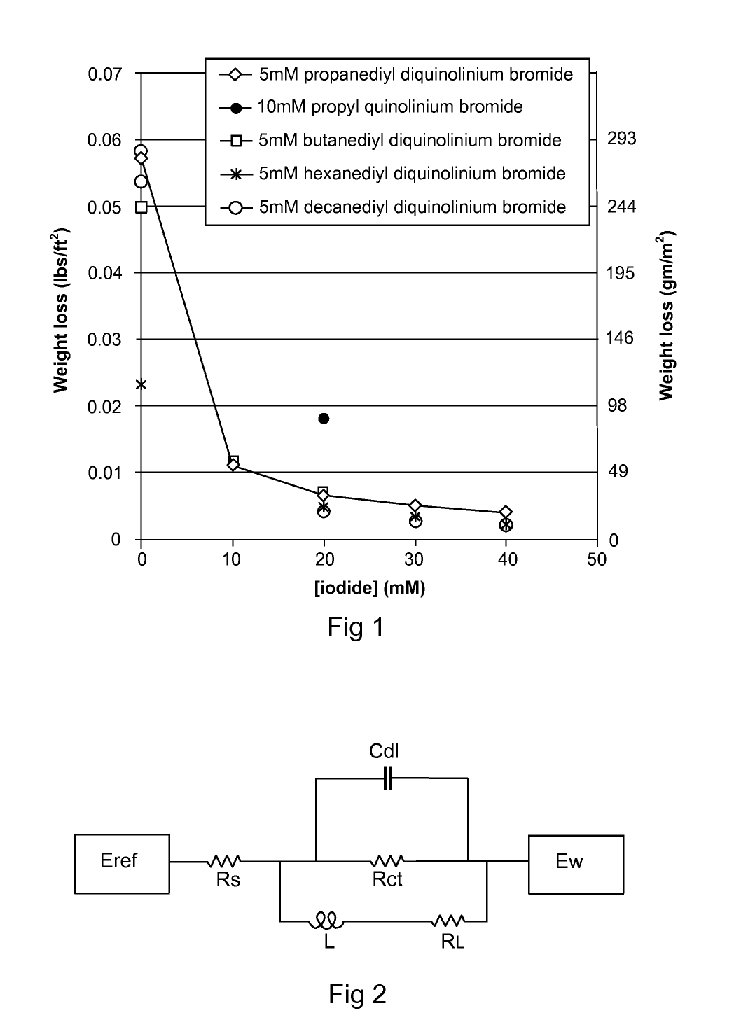

The results are shown graphically in FIG. 1. The data points for 5 mM propanediyl diquinolinium bromide are shown connected by a line. As is apparent, corrosion inhibition is enhanced by increasing concentrations of iodide. As shown, when 5 mM propanediyl diquinolinium bromide was used with 10 mM iodide, which is the equivalent quantity of iodide ion, the weight loss was reduced by a factor of about 5 compared to 5 mM propanediyl diquinolinium bromide without added iodide.

The same weight loss test was carried out on 22Cr125 steel with 5 mM concentration of propanediyl diquinolinium iodide without addition of any potassium iodide. The weight loss measured was almost the same as the weight loss measured with 5 mM propanediyl diquinolinium bromide and 10 mM potassium iodide, demonstrating that the enhanced corrosion protection in the presence of iodide results from the presence of this ion, and not merely from increased concentration of halide ions.

The corrosion inhibition was further improved as the length of the linking group increased from three to six and from six to ten carbon atoms. The comparative result for 10 mM propylquinolinium bromide with 20 mM iodide in Example 1 has been included in FIG. 1, with a filled circle as the data point.

### Example 3

Weight loss testing as in the previous examples was carried out on a range of steels using decanediyl diquinolinium bromide with varying concentrations of potassium iodide in solution. Results are set out in the following table which includes some results from previous examples.

| | | Total | Weight | Weight |
|---|---|---|---|---|
| | | iodide | loss | loss |
| Inhibitor in acid solution | Steel | concentration | (lbs/ft²) | (gm/m²) |
| 5 mM decanediyl di- | HS80 | none | 0.042 | 204 |
| quinolinium bromide | N80 | none | 0.025 | 122 |
| | 13Cr | none | 0.048 | 234 |
| | 22Cr125 | none | 0.057 | 278 |
| | HS80 | 20 mM | 0.0059 | 28.8 |
| | N80 | 20 mM | 0.0045 | 22.0 |
| | 13Cr | 20 mM | 0.0090 | 43.9 |
| | 22Cr125 | 20 mM | 0.0044 | 21.5 |
| | 22Cr125 | 30 mM | 0.0029 | 14.2 |
| | HS80 | 40 mM | 0.0050 | 24.4 |
| | N80 | 40 mM | 0.0033 | 16.1 |
| | 13Cr | 40 mM | 0.0061 | 29.8 |
| | 22Cr125 | 40 mM | 0.0023 | 11.2 |

These results show that the presence of iodide leads to a very considerable reduction in weight loss (i.e. enhanced corrosion protection). With all the steels, the presence of 20 mM iodide reduced the weight loss by a factor of four or more. There is some further improvement with higher concentrations of iodide.

### Example 4

The weight loss testing procedure as in previous examples was carried out using 5 mM butenediyldiquinolinium bromide (formula and preparation given previously) which has an olefinic unsaturation in the linking group and also 5 mM phenylenebis methylene diquinolinium bromide which has the structure below with an aromatic ring in the linking group.

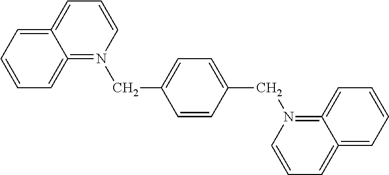

The weight loss results are given in the following table which also includes some results shown in FIG. 1.

| | | Total | Weight | Weight |
|---|---|---|---|---|
| | | iodide | loss | loss |
| Inhibitor in acid solution | Steel | conc. | (lbs/ft²) | (gm/m²) |
| 5 mM butanediyl di- | 22Cr125 | none | 0.050 | 244 |
| quinolinium bromide | | | | |
| 5 mM butenediyl di- | 22Cr125 | none | 0.057 | 278 |
| quinolinium bromide | | | | |
| 5 mM phenylenebismethylene | HS80 | none | 0.053 | 259 |
| di-quinolinium bromide | N80 | none | 0.035 | 171 |
| | 22Cr125 | none | 0.019 | 93 |
| | HS80 | 20 mM | 0.0097 | 47.3 |
| | N80 | 20 mM | 0.0048 | 23.4 |
| | 22Cr125 | 20 mM | 0.0043 | 21.0 |
| | HS80 | 40 mM | 0.0073 | 35.6 |
| | N80 | 40 mM | 0.0043 | 21.0 |

Once again, these results show that the presence of iodide leads to a very considerable reduction in weight loss (i.e. enhanced corrosion protection).

### Example 5

The weight loss testing procedure as in previous examples was also carried out with a di-isoquinolinium compound, namely with 5 mM hexanediyl di-isoquinolinium. The results, together with the results for the corresponding quinolinium compound is given in the following table.

| | | Total | Weight | Weight |
|---|---|---|---|---|
| | | iodide | loss | loss |
| Inhibitor in acid solution | Steel | conc. | (lbs/ft²) | (gm/m²) |
| 5 mM hexanediyl di- | 22Cr125 | none | 0.016 | 78.1 |
| isoquinolinium bromide | HS80 | 20 mM | 0.011 | 53.7 |
| | N80 | 20 mM | 0.0065 | 31.7 |
| | 22Cr125 | 20 mM | 0.0034 | 16.5 |
| | 22Cr125 | 30 mM | 0.0030 | 14.6 |
| | 22Cr125 | 40 mM | 0.0024 | 11.7 |
| 5 mM hexanediyl di- | 22Cr125 | none | 0.023 | 112 |
| quinolinium bromide | HS80 | 20 mM | 0.013 | 63.4 |
| | N80 | 20 mM | 0.0083 | 40.5 |
| | 22Cr125 | 20 mM | 0.0054 | 26.35 |
| | 22Cr125 | 30 mM | 0.0037 | 18.1 |
| | 22Cr125 | 40 mM | 0.0030 | 14.6 |

As can be seen, the isoquinolinium compound was even better than its quinolinium counterpart.

### Example 6

Corrosion inhibition by some of the above mentioned di-quinolinium and di-isoquinolinium compounds was also examined by Electrochemical Impedance Spectroscopy. The steel sample, subjected to corrosion, was in the form of a rotating cylinder electrode, that is to say it was in the form of a cylinder, partially immersed in the corrosive acid solution, and rotated at 2000 rpm. This electrode was made of 22Cr125 steel. The surface area in contact with acid solution was 1.88 cm². The rotation provided dynamic flow of the electrolyte relative to the steel, representing flow of acid over fixed tubing. The rotating cylinder was used as the working electrode of a three-electrode electrochemical cell. The other electrodes were a Ag/AgCl, KCl (3M) reference electrode and a graphite counter electrode. The electrolyte was the corrosive acid, which in these experiments was 4M hydrochloric acid solution containing the corrosion inhibitor being tested. The cell was maintained at 80° C. The impedance of the cell was determined using a potentiostat to measure current and phase when a 2 mV alternating potential was superimposed on direct bias potential (equal to the open circuit potential of the system) applied to the working electrode. This was done while the frequency of the alternating potential was varied in steps from 10 KHz to 0.1 Hz.

The experimental data was fitted to the theoretical impedance of an equivalent circuit shown in FIG. 2 where Ew is the working electrode, Eref is the reference electrode, Rs, Rct, and RL are the solution resistance, charge transfer resistance and the inductor resistance respectively, Cdl is the double layer capacitance and L is the inductor associated with the corrosion intermediates. Fitting the data, using a Nyquist plot allowed the charge transfer resistance to be determined. Charge transfer resistance is the parameter of interest because it is inversely proportional to the corrosion rate at the time of the test.

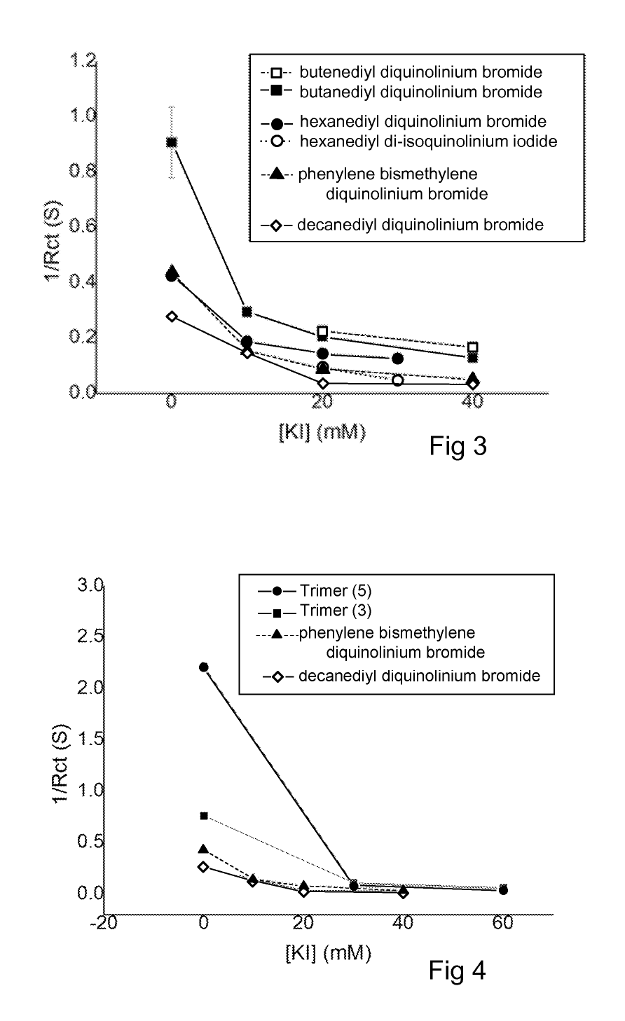

FIG. 3 is a plot of the reciprocal of charge transfer resistance for a number of compounds mentioned above, with varying iodide concentrations. Similarity to the weight loss data in previous examples was clearly apparent as was the enhancement of corrosion protection in the presence of iodide ion.

The compounds tested, as named herein, were the following:

butenediyl diquinolinium bromide (with double bond in linking group)
butanediyl quinolinium bromide
hexanediyl diquinolinium bromide
hexanediyl diisoquinolinium bromide
phenylenebis methylene diquinolinium bromide
decanediyl diquinolinium bromide

### Example 7

Electrochemical Impedance Spectroscopy was also used to examine corrosion inhibition by the two tris-quinolinium compounds referred to as (3) and (5) in the examples of synthesis given earlier. The results, at varying iodide concentrations, for these two compounds and also for two diquinolinium compounds are shown in FIG. 4. Yet again the presence of iodide ion greatly enhances corrosion protection.

This technique was also used to examine corrosion inhibition by the tris phenathridium compound shown as compound (7) in the examples of synthesis. The tests were carried out at 30 mM iodide and at 60 mM iodide. At both iodide concentrations, the tris phenanthridium inhibitor was as good as, or better than, the tris quinolinium compounds.

It will be appreciated that the embodiments and examples described in detail above can be modified and varied within the scope of the concepts which they exemplify. Features referred to above or shown in individual embodiments above may be used together in any combination as well as those which have been shown and described specifically. Accordingly, all such modifications are intended to be included within the scope of this disclosure as defined in the following claims.

### Example 8

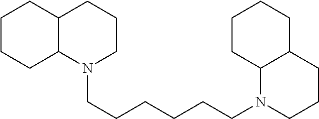 [SMILES: C(CCCN1CCCC2CCCCC21)CCN1CCCC2CCCCC21]

### 1,6-Bis (octahydroquinolin-1(2H)-yl)hexane

In the hydroquinoline dimer shown above, the two hydroquinoline sub-structures are linked by a linear hexyl chain.

(bisOHQyl)hexane contains two ring nitrogen atoms which convert to the protonated form under acid conditions—this reversible process leads to good acid solubility and insolubility (and low toxicity) under neutral pH conditions.

(bisOHQyl)hexane Synthesis:

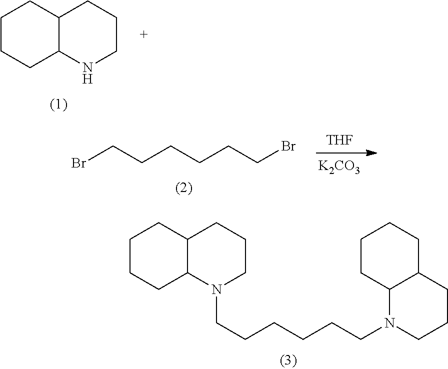

1,2,3,4-Tetrahydroquinoline (639 mg, 4.8 mmol), 1,6-dibromohexane (488 mg, 2 mmol) and potassium carbonate (800 mg, 5.8 mmol) were heated, in dimethylformamide at 90° C. for 17 hr. The suspension was filtered and the solid was washed with diethyl ether. Water was added to the filtrate, the aqueous phase was removed and extracted with diethyl ether. The organic phase was evaporated and residue applied to silica column and eluted with hexane to give 1,6-bis (octahydroquinolin-1(2H)-yl)hexane (3), 85 mg (12% yield).

1. A method of inhibiting corrosion of metal exposed to aqueous acidic solution comprising including in the solution:
at least one compound comprising two to five groups which are connected together and each contain a heterocyclic aromatic ring or ring system comprising at least one ring nitrogen atom which is quaternary in the aqueous acidic solution; and
at least one adsorption-intensifying anion which comprises at least one atom of atomic number of 52 or above and/or an anionic group conjugated with an aromatic ring or ring system;
wherein the concentration, in equivalents, of the at least one adsorption-intensifying anion is at least equal to the concentration, in equivalents, of the at least one ring nitrogen atom.

2. The method according to claim 1 wherein the at least one ring nitrogen atom is tertiary in aqueous neutral or alkaline solutions.

3. The method according to claim 1 wherein the concentration, in equivalents, of the at least one adsorption-intensifying anion is at least twice the concentration, in equivalents, of the at least one ring nitrogen atom.

4. The method according to claim 1 wherein the molar concentration of the at least one adsorption-intensifying anion is at least twice the concentration, in equivalents, of the at least one ring nitrogen atom.

5. The method according to claim 1 wherein the at least one adsorption-intensifying anion carries a single negative charge.

6. The method according to claim 1 wherein the at least one adsorption-intensifying anion comprises iodide.

7. The method according to claim 1 wherein the at least one adsorption-intensifying anion comprises an aromatic sulphonate.

8. The method according to claim 1 wherein the number of quaternary nitrogen-containing groups which are connected together is from two to four.

9. The method according to claim 1 wherein each of the quaternary nitrogen-containing groups contains only a single ring nitrogen atom.

10. The method according to claim 1 wherein each group is covalently bonded through its quaternary nitrogen atom to a linking group which comprises a plurality of carbon atoms.

11. The method according to claim 10 wherein the linking group comprises a chain of at least three carbon atoms.

12. The method according to claim 1 wherein at least one of the quaternary nitrogen-containing groups contains a heterocyclic ring or ring system which is any one of pyridine, pyridinium, quinoline, quinolinium, isoquinoline, isoquinolinium, anthradine, anthradinium, phenanthradine, phenanthradinium, tetrahydroquinoline, or decahydroquinoline.

13. The method according to claim 1 wherein each quaternary nitrogen-containing group contains a heterocyclic ring or ring system which is any one of pyridine, pyridinium, quinoline, quinolinium, isoquinoline, isoquinolinium, anthradine, anthradinium, phenanthradine, phenanthradinium, tetrahydroquinoline, or decahydroquinoline and each quaternary nitrogen-containing group is connected through the quaternary nitrogen atom to a linking group which comprises a plurality of carbon atoms.

14. The method according to claim 1 wherein the solution is an acidic solution with a pH below zero.

15. The method according to claim 14 wherein the solution is a matrix acidizing fluid.

16. The method according to claim 1 wherein the metal is steel tubing located within, connected to or delivering into a subterranean borehole and the method includes pumping the solution through the metal tubing.

17. The method according to claim 1 wherein the metal is duplex alloy steel.
